(19) United States
(12) Patent Application Publication
(10) Pub. No.: US 2024/0066273 A1
(43) Pub. Date: Feb. 29, 2024

(54) Extension Set Having Distally-Directed Extension Tube

(71) Applicant: Becton, Dickinson and Company, Franklin Lakes, NJ (US)

(72) Inventor: Jonathan Karl Burkholz, Salt Lake City, UT (US)

(21) Appl. No.: 18/238,613

(22) Filed: Aug. 28, 2023

Related U.S. Application Data
(60) Provisional application No. 63/401,899, filed on Aug. 29, 2022.

(51) Int. Cl. A61M 25/06; A61M 25/00; A61M 39/12

(52) U.S. Cl. CPC A61M 25/0606; A61M 25/0014; A61M 25/0097; A61M 39/12; A61M 2025/0253

(57) ABSTRACT

Provided herein is an apparatus including a connector having a distal port, a proximal port, a first lumen extending between the distal port and the proximal port, a side port arranged in the connector between the proximal coupler and the distal coupler, the side port configured to be in fluid communication with the first lumen, and a fluid conduit having a proximal end coupled to the side port and a distal end, the fluid conduit in fluid communication with the side port and configured to extend distally from the connector. The apparatus further includes a stabilization platform integrally formed with at least a part of the connector, the stabilization platform configured to be in contact with a patient, and an elongated arm configured to extend distally from the connector or the stabilization platform.

### CROSS-REFERENCE TO RELATED APPLICATION

The present application claims priority to U.S. Provisional Application No. 63/401,899 entitled “Extension Set Having Distally-Directed Extension Tube” file Aug. 29, 2022, the entire disclosure of which is hereby incorporated by reference in its entirety.

### BACKGROUND OF THE INVENTION

### Field of the Invention

The present disclosure relates generally to extension sets for intravenous (IV) catheter assemblies and, more specifically, to extension sets with features for improving blood collection through indwelling catheters.

### Description of Related Art

A vascular access device (VAD) may access peripheral vasculature of a patient. A VAD may be indwelling for short term (days), moderate term (weeks), or long term (months to years). VADs may be used for infusion therapy and/or for blood withdrawal.

A common type of VAD is an over-the-needle peripheral intravenous catheter (PIVC). Currently, there may be several limitations to the use of a PIVC for fluid infusion or blood draw. Among these limitations is that the smaller gauge of the blood collection device that is necessary to pass through the PIVC to the patient's vasculature can result in increased dwell times due to low flow rates. This increased dwell time can result in thrombogenesis and failure of the blood collection device. Accordingly, there is a need in the art for devices and systems that allow for the introduction of instruments into a PIVC with reduced risk of thrombogenesis.

In some instances, an extension set can be coupled to a VAD, to provide additional flexibility for medical professionals in terms of administering and/or withdrawing fluids from the VAD. There are times when current VADs, and consequently extension sets, are manipulated manually to enable blood draw; however, doing so in an uncontrolled way can increase incidence of dislodgement of the VAD from the vasculature, thus shortening the life of the catheter indwell. Accordingly, a need exists in the art for improved devices, systems, and methods of manipulating an indwelling catheter to free the catheter tip and allow for blood collection and fluid delivery therefrom or therethrough.

### SUMMARY OF THE INVENTION

Provided herein is an apparatus, including a connector having a distal port configured to be coupled to a hub of a catheter, a proximal port, a first lumen extending between the distal port and the proximal port, the lumen being in fluid communication with a second lumen defined by the catheter when the distal port is coupled to the hub, a side port arranged in the connector between the proximal coupler and the distal coupler, the side port configured to be in fluid communication with the first lumen, and a fluid conduit having a proximal end coupled to the side port and a distal end, the fluid conduit in fluid communication with the side port and configured to extend distally from the connector. The apparatus further includes a stabilization platform integrally formed with at least a part of the connector, the stabilization platform configured to be in contact with a patient when the distal coupler is coupled to the hub of the catheter to stabilize the connector and the catheter, and an elongated arm configured to extend distally from the connector or the stabilization platform.

In accordance with an aspect of the present invention, the elongated arm and the fluid conduit are configured to intersect one another at a location distal to the distal port of the connector.

In accordance with an aspect of the present invention, an anchor is configured to reversibly couple to the patient.

In accordance with an aspect of the present invention, the anchor is coupled to the fluid conduit.

In accordance with an aspect of the present invention, the anchor is arranged at an intersection of the elongated arm and the fluid conduit.

In accordance with an aspect of the present invention, the connector includes a second side port.

In accordance with an aspect of the present invention, a second fluid conduit is coupled to the second side port.

In accordance with an aspect of the present invention, the second fluid conduit is configured to extend proximally from the second side port.

In accordance with an aspect of the present invention, the elongated arm is a second fluid conduit and is in fluid communication with the second side port.

In accordance with an aspect of the present invention, the fluid conduit includes a luer adapter at the distal end thereof.

In accordance with an aspect of the present invention, the fluid conduit is coupled, at the distal end thereof, to a needless access connector.

Also provided herein is an apparatus, including a connector having a distal port configured to be coupled to a hub of a catheter, a proximal port, a first lumen extending between the distal port and the proximal port, the lumen being in fluid communication with a second lumen defined by the catheter when the distal port is coupled to the hub, a side port arranged in the connector between the proximal coupler and the distal coupler, the side port configured to be in fluid communication with the first lumen, and a fluid conduit having a proximal end coupled to the side port and a distal end, the fluid conduit in fluid communication with the side port and the second connector. The apparatus further includes a stabilization platform integrally formed with at least a part of the connector, the stabilization platform configured to be in contact with a patient when the distal coupler is coupled to the hub of the catheter to stabilize the connector and the catheter, and an elongated arm configured to extend distally from the connector or the stabilization platform.

In accordance with an aspect of the present invention, the fluid conduit is configured to extend proximally from the connector.

In accordance with an aspect of the present invention, a second connector arranged at the distal end of the fluid conduit.

In accordance with an aspect of the present invention, the second connector includes a distal port coupled to the distal end of the fluid conduit, a proximal port, a third lumen between the distal port and the proximal port, and a side port configured to be in fluid communication with the third lumen.

In accordance with an aspect of the present invention, a second fluid conduit having a proximal end is coupled to the second connector side port and a distal end, the second fluid conduit in fluid communication with the second connector side port, the second fluid conduit configured to extend distally from the second connector.

In accordance with an aspect of the present invention, the elongated arm and the second fluid conduit are configured to intersect one another at a location distal to the distal port of the connector.

In accordance with an aspect of the present invention, an anchor is configured to reversibly couple to the patient.

In accordance with an aspect of the present invention, the anchor is coupled to the second fluid conduit.

In accordance with an aspect of the present invention, the anchor is arranged at an intersection of the elongated arm and the second fluid conduit.

In accordance with an aspect of the present invention, the second fluid conduit includes a luer adapter at the distal end thereof.

In accordance with an aspect of the present invention, the second fluid conduit is coupled, at the distal end thereof, to a needless access connector.

In accordance with yet another embodiment of the present invention, a system includes a catheter and an adapter. The adapter includes a connector having a distal port configured to be coupled to a hub of a catheter, a proximal port, a first lumen extending between the distal port and the proximal port, the lumen being in fluid communication with a second lumen defined by the catheter when the distal port is coupled to the hub, a side port arranged in the connector between the proximal coupler and the distal coupler, the side port configured to be in fluid communication with the first lumen, and a fluid conduit having a proximal end coupled to the side port and a distal end, the fluid conduit in fluid communication with the side port and configured to extend distally from the connector. Also included is a stabilization platform integrally formed with at least a part of the connector, the stabilization platform configured to be in contact with a patient when the distal coupler is coupled to the hub of the catheter to stabilize the connector and the catheter, and an elongated arm configured to extend distally from the connector or the stabilization platform.

### BRIEF DESCRIPTION OF THE DRAWINGS

FIG. 1 is a perspective view of a non-limiting embodiment of a prior art extension set;

FIGS. 2A-2B are perspective views of non-limiting embodiments of extension sets described herein;

FIGS. 3A-3B are perspective views of non-limiting embodiments of extension sets described herein; and

FIGS. 4A-4B are perspective views of non-limiting embodiments of extension sets described herein.

### DESCRIPTION OF THE INVENTION

The following description is provided to enable those skilled in the art to make and use the described embodiments contemplated for carrying out the invention. Various modifications, equivalents, variations, and alternatives, however, will remain readily apparent to those skilled in the art. Any and all such modifications, equivalents, variations, and alternatives are intended to fall within the spirit and scope of the present invention.

For purposes of the description hereinafter, the terms “upper”, “lower”, “right”, “left”, “vertical”, “horizontal”, “top”, “bottom”, “lateral”, “longitudinal”, and derivatives thereof shall relate to the invention as it is oriented in the drawing figures. However, it is to be understood that the invention may assume various alternative variations, except where expressly specified to the contrary. It is also to be understood that the specific devices illustrated in the attached drawings, and described in the following specification, are simply exemplary embodiments of the invention. Hence, specific dimensions and other physical characteristics related to the embodiments disclosed herein are not to be considered as limiting.

It should be understood that any numerical range recited herein is intended to include all values and sub-ranges subsumed therein. For example, a range of “1 to 10” is intended to include all sub-ranges between (and including) the recited minimum value of 1 and the recited maximum value of 10, that is, having a minimum value equal to or greater than 1 and a maximum value equal to or less than 10.

U.S. Patent Application Publication No. 2019/0160275 is incorporated herein by reference in its entirety.

Provided herein are devices and systems for securing and/or stabilizing extension sets used with indwelling catheters, such as peripheral intravenous catheters (PIVCs). While certain devices (e.g., blood draw devices) are discussed below in terms of devices that may be used with PIVCs and extension sets, as described herein, those of skill will appreciate that any number of different devices for introducing an instrument, including instruments ranging from tubes, probes, sensors, wiring, fiber optics, guidewires, etc., may be used within the scope of the present disclosure.

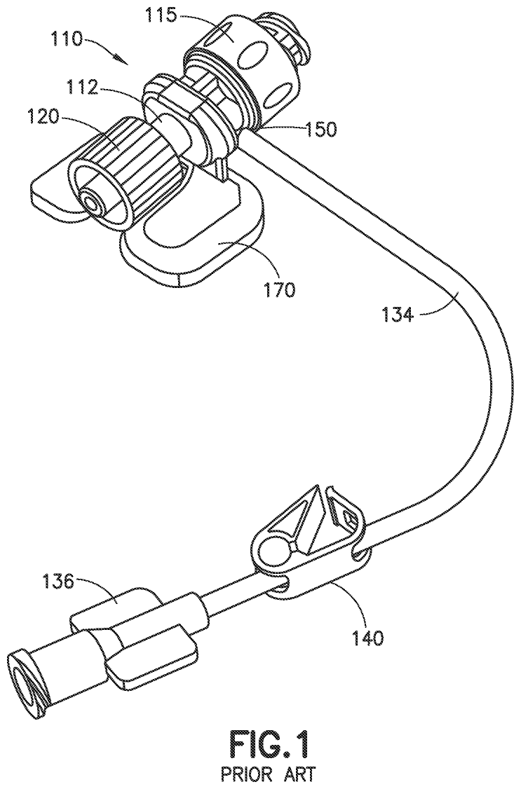

Referring now to FIG. 1, shown is a prior art extension set, including a stabilizing connector 110 and fluid conduit 134. The stabilizing connector 110 is configured to be placed in contact with the skin of a patient at or near an insertion site of an indwelling or placed vascular access device (VAD), such as those described above and known to those of skill in the art. The stabilizing connector 110 is configured to couple to and/or otherwise engage the VAD. Once coupled to a VAD, the stabilizing connector 110 can be secured to the skin of the patient (e.g., via medical tape, a clear sterile barrier such as Tegaderm™, and/or the like), which in turn secures and/or stabilizes at least a portion of the VAD relative to the patient, as described in further detail herein.

The stabilizing connector 110 can be any suitable shape, size, and/or configuration. In the illustrated non-limiting embodiment, the stabilizing connector 110 has a connector portion 112 and a stabilization portion 170. In some embodiments, the stabilizing connector 110 can be configured as a combination of one or more stabilization device(s) and an extension set. Each of the connector portion 112 and/or the stabilization portion 170 can be arranged in any suitable manner to facilitate at least one of the functions of providing stabilization to one or more devices (e.g., a VAD or the like) and/or at least one of the functions of providing an extension set for use with a VAD.

The connector portion 112 has a proximal port with a coupler 115, a distal port with a coupler 120, and defines at least one lumen (not shown) extending through or otherwise in fluid communication with the couplers 115 and 120. The proximal coupler 115 and/or the distal coupler 120 can be, for example, male or female luer locks and/or any other suitable coupler. The proximal coupler 115 can be physically and fluidically coupled to any suitable medical device such as those described above. The distal coupler 120 can be physically and fluidically coupled to, for example, a VAD or the like such that the lumen of the connector portion 112 is at least selectively in fluid communication with the VAD and/or a portion of the body in which the VAD is at least partially disposed. In some embodiments, the lumen of the connector portion 112 can be substantially straight and/or can allow for a substantially straight line of sight therethrough. In some embodiments, an inner surface of the connector portion 112 can be configured to provide alignment, guidance, centering, etc., to an object or device (e.g., a blood draw catheter or the like, as described above) being advanced therethrough.

In some embodiments, the couplers 115 and/or 120 can be arranged and/or configured to accept a click-to-connect coupling (e.g., a Click-Lock-Snap™ connection), a threaded coupling, a luer connection, and/or the like. In some non-limiting embodiments, the couplers 115 and/or 120 may include, for example, a spin collar or the like and/or may otherwise be configured to form relatively easy, secure, and fluid tight connections. In some embodiments, the couplers 115 and/or 120 can be a needleless or needle-free connector, an independent connector, and/or a swappable connector, and/or can be compatible with any suitable valve and/or seal (e.g., a valve used in a luer lock, a needle-free connector (NFC) (also known as a needless access connector, (NAC)), a split septum, and/or the like). As a specific, non-limiting example, the proximal port 115 can be configured as an NFC or the like and may include an NFC valve (e.g., similar to or the same as known needle-free connectors and/or known NFC valves).

In some embodiments, the distal coupler 120 can be configured to couple to, for example, a VAD such that engagement allows a tapered portion thereof to slip into a hub or the like to establish hemostasis, and can include a floating collar or the like such that the connector portion 112 remains coupled to the VAD during manipulation of one or more devices coupled thereto. In some embodiments, the couplers 115 and/or 120 can be configured to be compatible with any suitable known coupler or connector. In some embodiments, the coupler 120 can be or can include, for example, a floating male luer spin collar, a rotating male luer lock collar, or the like. Moreover, in some embodiments, the couplers 115 and/or 120 can include protective caps and/or the like that are removably coupled to the couplers 115 and 120. Such protective caps can be slip or friction fit or can be coupled via a threaded coupling.

The connector portion 112 also includes and/or defines one or more additional ports, such as a side port 150. Side port 150 can be included in and/or can be a part of the connector portion 112, the proximal coupler 115, the distal coupler 120, and/or a combination thereof. For example, side port 150 may be distal to the proximal coupler 115. In other embodiments, however, a connector portion 112 can include and/or define a side port 150 that is formed by a structure or feature forming a proximal coupler. In some instances, positioning side port 150 in a desired position along a length of the connector portion 112, between the couplers 115 and 120, can allow for a reduced length of the connector portion 112 and/or can facilitate flushing and/or fluid transfer via side port 150.

In some embodiments, the arrangement of side port 150 can be such that the connector portion 112 forms, for example, a Y-connector or a T-connector. More particularly, side port 150 can be disposed substantially perpendicular (e.g., about 90 degrees) to the lumen of the connector portion 112 and near or adjacent the proximal coupler 115. In non-limiting embodiments, side port 150 extends from connector portion 112 at an angle, relative to the lumen between distal coupler 120 and proximal coupler 115, that is not 90 degrees (e.g., side port 150 extends at an angle of, for example and without limitation, 15-165 degrees, all values and subranges therebetween inclusive). In non-limiting embodiments, more than one side port 150 is included in connector portion 112.

In some embodiments, a position of the port 150 can be shifted along the connector portion 112 in a desired manner to enable use of a shorter proximal connector (e.g., the proximal coupler 115). In some embodiments, the port 150 and/or the fluid conduit 134 coupled thereto can be and/or can form at least a portion of a fluid line that can be used to deliver fluid, remove fluid, flush fluid, and/or the like. In such embodiments, for example, an arrangement in which side port 150 is disposed adjacent to the proximal coupler 115 can enable flushing of the proximal coupler 115, any valve(s) included therein, and/or a space between any valve(s) and an inner surface of the connector portion 112 (e.g., defining at least a portion of the lumen). In other embodiments, the connector portion 112 can be a single port connector that does not include the port 150 (and that can be flushed, for example, through the proximal port 115).

Side port 150 defines a lumen (not shown) that is in fluid communication with the lumen between distal coupler 120 and proximal coupler 115. In other words, the connector portion 112 and/or the port 150 can include and/or define a first lumen (e.g., between distal coupler 120 and proximal coupler 115) and a second lumen (e.g., lumen defined by side port 150 and fluid conduit 134). As such, side port 150 can provide access to the lumen between distal coupler 120 and proximal coupler 115, which in turn can provide access to a device (e.g., a VAD) that is coupled to the distal coupler 120 and/or can provide access to a portion of the body in which the VAD is at least partially disposed.

As shown in FIG. 1, side port 150 may be coupled to fluid conduit 134 that is in fluid communication with the lumen of the port 150. In some embodiments, the connector 110 and/or any suitable portion thereof can include one or more features configured to manage and/or direct at least a portion of the fluid conduit 134 extending from side port 150. As shown, an end portion of the fluid conduit 134 (e.g., an end portion opposite side port 150) can include and/or can be coupled to an attachment device, coupler, connector, port, luer, and/or the like. For example, in the embodiment shown in FIG. 1, fluid conduit 134 includes a luer 136 at a distal end thereof, where the luer 136 may be configured to couple to any suitable device, such as a fluid source, a fluid collection device, an evacuated container, a pump, a syringe, and/or any other suitable device. Moreover, in some embodiments, the connector 110 can include a clamp 140 coupled to the fluid conduit 134 and configured to selectively engage the fluid conduit 134 to constrict, crimp, clamp, and/or otherwise occlude a lumen defined by the fluid conduit 134 to limit and/or substantially prevent a flow of fluid therethrough. Although the clamp 140 is shown as being separate from luer 136, in other embodiments, at least a portion of the clamp 140 can be integrated into the luer 136.

In some embodiments, the connector 110 can be configured for use with and/or configured to control a pressure or flow rate through at least a portion of the connector portion 112 and/or the lumen thereof. In some embodiments, the connector portion 112 can include and/or can accept one or more backflow preventers and/or valves (e.g., anti-reflux valves, check valves, split septums, and/or the like), one or more pressure regulators, and/or any other suitable flow control device.

The stabilization portion 170 is coupled to the connector portion 112 and is configured to be placed in contact with a portion of a patient (e.g., the skin of the patient) at or near an insertion site associated with the VAD (or other similar device). The stabilization portion 170 can be any suitable shape, size, and/or configuration. For example, in some embodiments, the stabilization portion 170 can be and/or can form a base structure that is angled, tapered, flared, curved, rounded, and/or the like. In some embodiments, the stabilization portion 170 can have a base surface (or bottom surface) that has a contour and/or shape that is generally concave. In some embodiments, the concave contour and/or shape of the base surface can be based at least in part on a curvature and/or shape of a portion of the patient's anatomy. In some embodiments, forming the contour and/or shape of the base surface to be similar to and/or at least partially based on the curvature and/or angle of an IV insertion site of a patient, for example, can increase a surface area of the stabilization portion 170 (e.g., base surface) that is in contact with the skin of the patient, which in turn can increase the stability of the stabilizing connector 110 and reduce a pressure associated with the stabilizing connector 110 when secured to the skin of the patient, as described in further detail herein.

In some embodiments, the base surface of the stabilization portion 170 may be selectively formed from one or more materials (e.g., a relatively hard material and/or a relatively soft material), configured to provide both stabilization and comfort. In some embodiments, the stabilization portion 170 can be configured to provide increased stabilization to a given or desired portion (e.g., a proximal portion, a distal portion, one or more side portions, and/or the like). In some embodiments, the stabilization portion 170 can be reconfigurable, which can allow a user to selectively control an amount of stabilization provided by the stabilization portion 170. Moreover, in some such embodiments, a user can reconfigure (e.g., bend, flex, deform, conform, stretch, break, cut, add to, etc.) one or more portions of the stabilization portion 170 to, for example, control an amount or manner of stabilization, to conform at least a portion of the stabilization portion 170 to the contours of a specific patient, to reduce or substantially prevent pressure points, and/or the like.

The base surface of stabilization portion 170 may include one or more contours, recesses, notches, cutouts, channels, etc., (referred to herein as a “recess”) configured to reduce an amount of force exerted by the stabilization portion 170 on or more veins and/or arteries of the patient (e.g., the vein in which the VAD is disposed), which might otherwise result in an occlusion of and/or a reduced flow rate through at least a portion of the vasculature. The contour and/or shape of the base surface can be based, for example, at least in part on a curvature and/or shape of a portion of the patient's anatomy. In some embodiments, at least one of the one or more recesses can substantially extend between a proximal edge and a distal edge of the base surface.

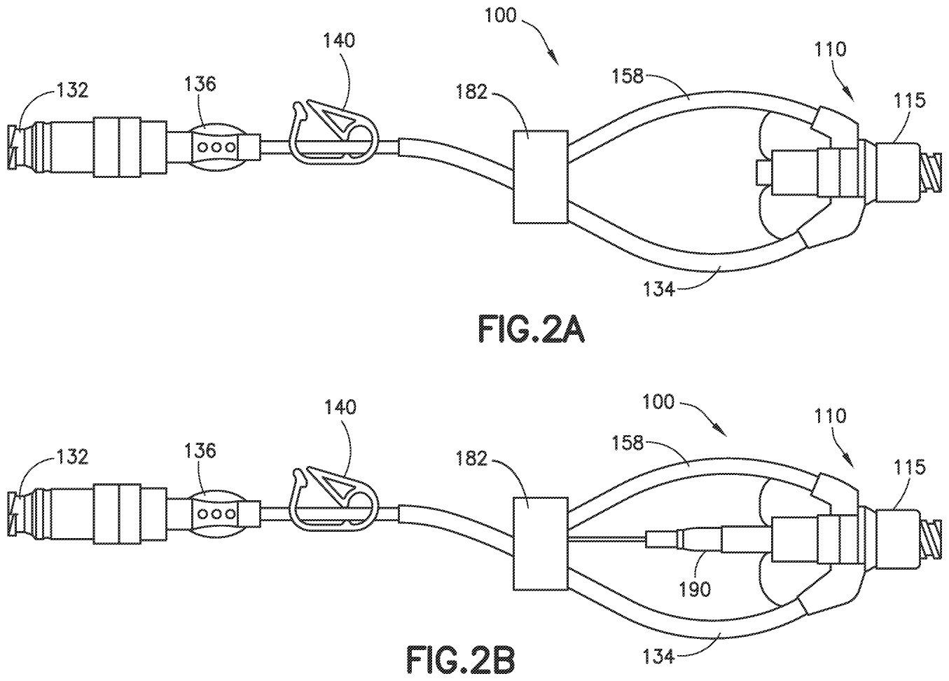

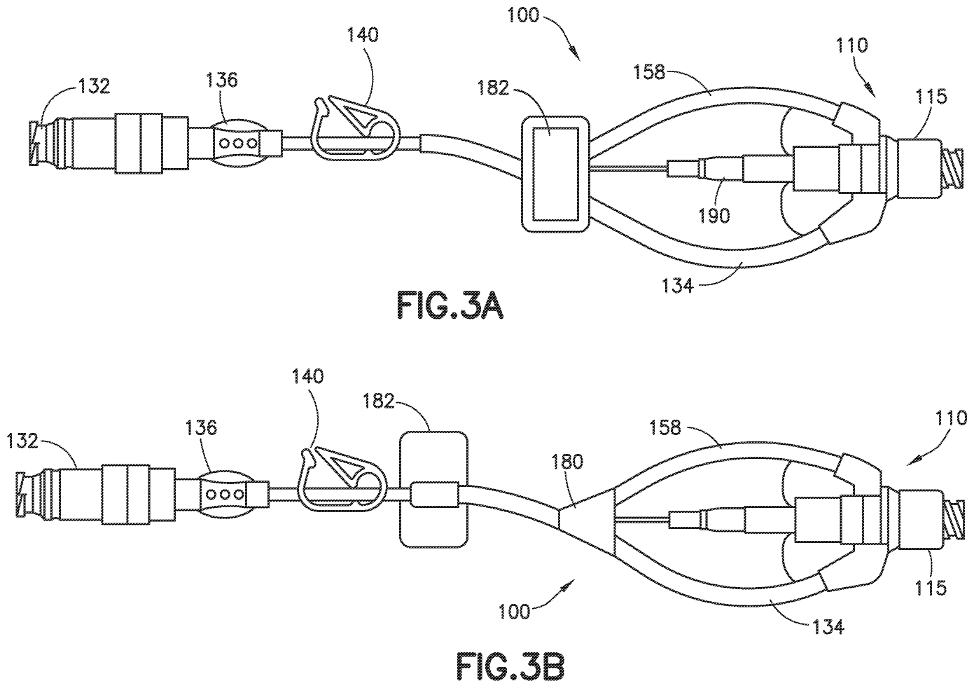

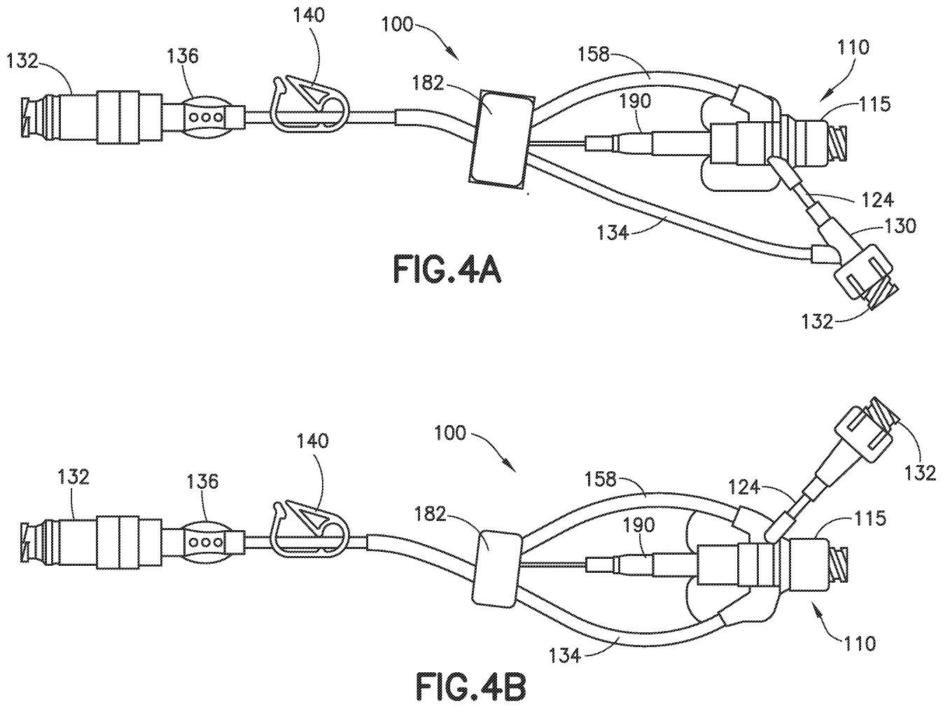

Turning to FIGS. 2A-4B, shown are non-limiting embodiments of an apparatus 100 including a stabilizing connector 110, a fluid conduit 134, and one or more elongated arms 158. One or more elongated arms 158 may be coupled to connector portion 112 and/or stabilization portion 170. Fluid conduit 134 and/or one or more arms 158 may be arranged on opposite sides of stabilizing connector 110, and may be configured to extend distally (for example, as shown in FIGS. 2A-4B) from stabilized connector 110. In non-limiting embodiments, fluid conduit 134 and one or more elongated arms 158 extend distally along an arc, thereby intersecting distally of distal coupler 120. At a point where fluid conduit 134 and one or more arms 158 intersect, an anchor 182 may be arranged, to couple fluid conduit 134 and one or more elongated arms 158 (for example, as in FIGS. 2A, 2B, 3A, 4A, and 4B). As described above, fluid conduit 134 may include, at distal end thereof, a connector, such as luer connecter 136 and/or a NFC/NAC 132. Those of skill will appreciate that NFCs/NACs 132 as described herein, may be integrated into conduits and/or connectors, or may be removably coupled to such structures. As shown in FIG. 2B (as well as FIGS. 3A-4B), apparatus 100 may be coupled to a catheter 190 through distal coupler 120. Apparatus 100 may further include a clamp 140 arrangeable anywhere along the length of fluid conduit 134, as described above.

One or more elongated arms 158 may be formed of any suitable material. While not wishing to be bound by the theory, elongated arm(s) 158 with a material having a substantially similar elasticity to materials typically used in fluid conduits, such as fluid conduit 134, may be preferable to minimize twisting of stabilized connecter 110 when a distally-directed force is applied to one or more elongated arms 158 and/or fluid conduit 134. By arranging one or more elongated arms 158 and/or fluid conduit 134 in the manner shown in the accompanying figures, force can be applied to stabilized connector 110 and, consequently, catheter 190, whereby catheter 190 is pulled further into the vasculature, rather than inadvertently being pulled out of the vasculature if, for example, fluid conduit 134 extended proximally.

The non-limiting embodiments of FIGS. 2A-4B show various possible configurations of apparatus 100, but those of skill will appreciate that these should not be considered limiting. For example, while the illustrated embodiments exemplify a single elongated arm 158, those of skill will appreciate that a plurality of elongated arms 158 may be coupled to connector portion 112 and/or stabilization portion 170. Further, in non-limiting embodiments, elongated arm(s) 158 may also be fluid conduits, coupled to one or more side ports 150 of connector portion 112. In non-limiting embodiments, apparatus 100 includes two elongated arms 158, two fluid conduits 134, or one elongated arm 158 and one fluid conduit 134.

With continuing reference to the accompanying figures, anchor 182 may be configured to be reversibly coupleable to the patient. For example, anchor 182 may include a backing on a skin-facing surface that includes an adhesive. Suitable adhesives for reversibly coupling components to a patient's skin are known to those of skill in the art. Anchor 182 may be formed of any suitable material, for example flexible materials, soft materials, semi-rigid materials, or rigid materials. Anchor 182 may also include one or more visible indicia, conveying information to healthcare professionals, such as catheter gauge, length, and/or information relating to one or more procedures to be performed through stabilized connector 110.

Turning to FIGS. 3A-3B, shown are non-limiting embodiments of arrangements of anchor 182. As shown in FIG. 3A, anchor 182 may be arranged at an intersection of one or more elongated arms 158 and fluid conduit 134. In non-limiting embodiments (for example, as shown in FIG. 3B), anchor 182 is arranged on fluid conduit 134, and a separate junction 180 is included that joins, reversibly or irreversibly, one or more elongated arms 158 and fluid conduit 134.

Turning to FIGS. 4A-4B, shown are non-limiting embodiments of arrangements of fluid conduit 134. As shown in FIG. 4A, a first fluid conduit 124 may be coupled, at a proximal end thereof, to side port 150. First fluid conduit 124 may be coupled, at a distal end thereof, to a connecter 130, which may be integral or connectable thereto to a NFC/NAC 132. In non-limiting embodiments, connecter 130 may include a further side port, to which a second fluid conduit 134 may be coupled. As may be appreciated from FIG. 4A, first fluid conduit 134 extends proximally from connector portion 112. Accordingly, in order to provide the intersection of one or more elongated arms 158 and fluid conduit 134, fluid conduit 134 extends distally from connector 130. With regard to FIG. 4B, connector portion 112 includes a plurality of side ports 150. Fluid conduit 134 extends distally from a side port 150, as described herein above. Another fluid conduit 124, having at a distal end thereof a connector and/or NFC/NAC 132, is provided in the illustrated non-limiting embodiment, extending proximally from another side port 150.

Although the present disclosure has been described in detail for the purpose of illustration based on what is currently considered to be the most practical and preferred embodiments or aspects, it is to be understood that such detail is solely for that purpose and that the present disclosure is not limited to the disclosed embodiments or aspects, but, on the contrary, is intended to cover modifications and equivalent arrangements that are within the spirit and scope of the appended claims. For example, it is to be understood that the present disclosure contemplates that, to the extent possible, one or more features of any embodiment may be combined with one or more features of any other embodiment.

The invention claimed is:

1. An apparatus comprising:
a connector having:
a distal port configured to be coupled to a hub of a catheter;
a proximal port;
a first lumen extending between the distal port and the proximal port, the lumen being in fluid communication with a second lumen defined by the catheter when the distal port is coupled to the hub;
a side port arranged in the connector between the proximal coupler and the distal coupler, the side port configured to be in fluid communication with the first lumen; and
a fluid conduit having a proximal end coupled to the side port and a distal end, the fluid conduit in fluid communication with the side port and configured to extend distally from the connector;
a stabilization platform integrally formed with at least a part of the connector, the stabilization platform configured to be in contact with a patient when the distal coupler is coupled to the hub of the catheter to stabilize the connector and the catheter; and
an elongated arm configured to extend distally from the connector or the stabilization platform.

2. The apparatus of claim 1, wherein the elongated arm and the fluid conduit are configured to intersect one another at a location distal to the distal port of the connector.

3. The apparatus of claim 1, further comprising an anchor configured to reversibly couple to the patient.

4. The apparatus of claim 3, wherein the anchor is coupled to the fluid conduit.

5. The apparatus of claim 3, wherein the anchor is arranged at an intersection of the elongated arm and the fluid conduit.

6. The apparatus of claim 1, wherein the connector comprises a second side port.

7. The apparatus of claim 6, further comprising a second fluid conduit coupled to the second side port.

8. The apparatus of claim 7, wherein the second fluid conduit is configured to extend proximally from the second side port.

9. The apparatus of claim 6, wherein the elongated arm is a second fluid conduit and is in fluid communication with the second side port.

10. The apparatus of claim 1, wherein the fluid conduit comprises a luer adapter at the distal end thereof.

11. The apparatus of claim 1, wherein the fluid conduit is coupled, at the distal end thereof, to a needless access connector.

12. An apparatus comprising:
a connector having:
a distal port configured to be coupled to a hub of a catheter;
a proximal port;
a first lumen extending between the distal port and the proximal port, the lumen being in fluid communication with a second lumen defined by the catheter when the distal port is coupled to the hub;
a side port arranged in the connector between the proximal coupler and the distal coupler, the side port configured to be in fluid communication with the first lumen; and
a fluid conduit having a proximal end coupled to the side port and a distal end, the fluid conduit in fluid communication with the side port and the second connector;
a stabilization platform integrally formed with at least a part of the connector, the stabilization platform configured to be in contact with a patient when the distal coupler is coupled to the hub of the catheter to stabilize the connector and the catheter; and
an elongated arm configured to extend distally from the connector or the stabilization platform.

13. The apparatus of claim 12, wherein the fluid conduit is configured to extend proximally from the connector.

14. The apparatus of claim 12, further comprising a second connector arranged at the distal end of the fluid conduit.

15. The apparatus of claim 14, wherein the second connector comprises a distal port coupled to the distal end of the fluid conduit, a proximal port, a third lumen between the distal port and the proximal port, and a side port configured to be in fluid communication with the third lumen.

16. The apparatus of claim 15, further comprising a second fluid conduit having a proximal end coupled to the second connector side port and a distal end, the second fluid conduit in fluid communication with the second connector side port, the second fluid conduit configured to extend distally from the second connector.

17. The apparatus of claim 16, wherein the elongated arm and the second fluid conduit are configured to intersect one another at a location distal to the distal port of the connector.

18. The apparatus of claim 17, further comprising an anchor configured to reversibly couple to the patient.

19. The apparatus of claim 18, wherein the anchor is coupled to the second fluid conduit.

20. The apparatus of claim 18, wherein the anchor is arranged at an intersection of the elongated arm and the second fluid conduit.

21. The apparatus of claim 16, wherein the second fluid conduit comprises a luer adapter at the distal end thereof.

22. The apparatus of claim 16, wherein the second fluid conduit is coupled, at the distal end thereof, to a needless access connector.

23. A system comprising:
a catheter; and
an adapter, the adapter comprising:
a connector having:
a distal port configured to be coupled to a hub of a catheter;
a proximal port;
a first lumen extending between the distal port and the proximal port, the lumen being in fluid communication with a second lumen defined by the catheter when the distal port is coupled to the hub;
a side port arranged in the connector between the proximal coupler and the distal coupler, the side port configured to be in fluid communication with the first lumen; and
a fluid conduit having a proximal end coupled to the side port and a distal end, the fluid conduit in fluid communication with the side port and configured to extend distally from the connector;
a stabilization platform integrally formed with at least a part of the connector, the stabilization platform configured to be in contact with a patient when the distal coupler is coupled to the hub of the catheter to stabilize the connector and the catheter; and
an elongated arm configured to extend distally from the connector or the stabilization platform.
